(19) United States
(12) Patent Application Publication
(10) Pub. No.: US 2013/0045256 A1
(43) Pub. Date: Feb. 21, 2013

(54) SHAVE PREPARATIONS COMPRISING AN ANTI-IRRITATION AGENT

(71) Applicant: James Robert Schwartz, West Chester, OH (US)

(72) Inventor: James Robert Schwartz, West Chester, OH (US)

(21) Appl. No.: 13/587,217

(22) Filed: Aug. 16, 2012

Related U.S. Application Data
(60) Provisional application No. 61/524,063, filed on Aug. 16, 2011.

(51) Int. Cl. A61K 8/58; A61K 31/555; A61Q 9/02; A61K 8/02

(52) U.S. Cl. 424/401; 424/73

(57) ABSTRACT

A shave preparation comprising water; one or more lipophilic skin conditioning agents; one or more thickening agents; one or more emulsifying agents; and one or more lubricants, and an anti-irritation agent such as zinc pyrithione.

### CROSS REFERENCE TO RELATED APPLICATION

This application claims the benefit of Provisional Application Ser. No. 61/524,063, filed Aug. 16, 2011, the contents of which are incorporated by reference herein.

### BACKGROUND OF THE INVENTION

Shave preparations are known in the art. However, problems remain with these preparations. Irritation and lack of shave closeness remain problems of these preparations. Non-limiting examples of known shave preparations include WO 93/18740, GB 02236760, U.S. Pat. No. 3,072,536, and U.S. Pat. No. 4,585,650, and US Patent Publ. No. 2010/0272667. Improved shave preparations that address one or more of these problems are desired in the art. This invention addresses these needs.

### SUMMARY OF THE INVENTION

One aspect of the invention provides for a shave preparation comprising water; one or more lipophilic skin conditioning agents; one or more thickening agents; one or more emulsifying agents; and one or more lubricants and an anti-irritation agent comprising a zinc pyrithione, a zinc salt, or a combination thereof.

Another aspect of the invention provides for a shave preparation comprising water; one or more lipophilic skin conditioning agents; one or more thickening agents; one or more emulsifying agents; and one or more lubricants, an anti-irritation agent comprising a zinc pyrithione, a zinc salt, or a combination thereof, wherein the shave preparation is substantially free of a gel network.

Yet another aspect of the invention provides for a shave preparation comprising: at least 60% water; from about 2% to about 4% of one or more lipophilic skin conditioning agents; from about 0.25% to about 3% of one or more thickening agents; from about 0.25% to about 3% of one or more emulsifying agents; and from about 0.25% to about 3% of one or more lubricants; from about 0.25% to about 2.5% of an anti-irritation agent wherein said anti-irritation agent comprises zinc pyrithione, and wherein the shave preparation is substantially free of a gel network.

### DETAILED DESCRIPTION OF THE INVENTION

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

The current invention provides for shave preparations having desirable properties that improve the shave experience. Without wishing to be bound by theory, it is believed that the compositions within the shave care preparations of the present invention interact synergistically to provide for an enhanced experience.

### Anti-irritation Agent

In one embodiment the shave preparation composition of the present invention further comprises an anti-irritation agent. The anti-irritation agent can be pyrithione or a polyvalent metal salt of pyrithione, or a mixture thereof. Any form of polyvalent metal pyrithione salts may be used, including platelet and needle structures. Preferred salts for use herein include those formed from the polyvalent metals magnesium, barium, bismuth, strontium, copper, zinc, cadmium, zirconium and mixtures thereof, more preferably zinc. Even more preferred for use herein is the zinc salt of 1-hydroxy-2-pyridinethione (known as “zinc pyrithione” or “ZPT”); more preferably ZPT in platelet particle form, wherein the particles have an average size of up to about 20 μm, preferably up to about 5 μm, more preferably up to about 2.5 μm.

The platelet ZPT includes a median particle diameter of about 0.5 microns to about 10, alternatively about 1 to about 5 microns, and alternatively about 3 microns; a mean particle diameter of about 0.5 to about 10 microns, alternatively about 1 to about 5 microns, alternatively about 2 to about 4 microns, and alternatively about 3 microns, and a thickness of about 0.6 to about 15 microns, alternatively about 0.6 to 1 micron, alternatively about 0.6 to about 0.8, and alternatively about 0.6 to about 0.7 microns. The platelet ZPT can also have a span of less than about 5, and alternatively about 1.

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

Preferred embodiments include from 0.01% to 5% of an anti-irritation agent; alternatively from 0.05% to 2%, alternatively from 0.1% to 1%, alternatively from 0.2% to about 0.7%, alternatively about 0.5%.

The composition of the present invention optionally includes an effective amount of a zinc salt. Preferred embodiments of the present invention include an effective amount of a zinc salt having an aqueous solubility within the composition of less than about 25%, by weight, at 25° C., more preferably less than about 20%, more preferably less than about 15%. Preferred embodiments of the present invention include from 0.001% to 10% of a zinc salt, more preferably from 0.01% to 5%, more preferably still from 0.1% to 3%. In a preferred embodiment, the zinc salt has an average particle size of from 100 nm to 30 μm.

Examples of zinc salts useful in certain embodiments of the present invention include the following: Zinc aluminate, Zinc carbonate, Zinc oxide and materials containing zinc oxide (i.e., calamine), Zinc phosphates (i.e., orthophosphate and pyrophosphate), Zinc selenide, Zinc sulfide, Zinc silicates (i.e., ortho- and meta-zinc silicates), Zinc silicofluoride, Zinc Borate, Zinc hydroxide and hydroxy sulfate, zinc-containing layered materials and combinations thereof.

In embodiments having an anti-irritation agent and a zinc salt, the ratio of zinc salt to anti-irritation agent is preferably from 5:100 to 5:1; more preferably from about 2:10 to 3:1; more preferably still from 1:2 to 2:1.

Those of skill in the art will understand that the anti-irritation agent of the present invention can also have other benefits which may be desirable from a skin care composition, including but not limited to malodor control and/or anti-bacterial benefits depending on whether the composition is left on skin or rinsed off.

Without intending to be bound by theory, it is believed that these anti-irritation agents can provide various benefits including reduction or control of irritation as well as certain malodor control. In one embodiment, the composition further comprises other agents such as malodor control agents. The malodor active of the present invention is capable of providing an antimicrobial benefit. Such malodor actives are capable of destroying microbes, preventing the development of microbes or preventing the pathogenic action of microbes. A safe and effective amount of a malodor active may be added to the intimate cleansing product, at from about 0.001% to about 10%, or from about 0.01% to about 5%, or from about 0.05% to about 2%, or from about 0.1% to about 1%, or from about 0.3% to about 0.7%, or about 0.5% by weight of the composition.

Examples of malodor actives include β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, clotrimazole, and mixtures thereof.

### Water

The shave preparations of the current invention comprise water. In one embodiment, the shave preparation comprises at least about 60% by weight water. In an alternate embodiment, the shave preparation comprises at least about 70% by weight water. In an alternate embodiment, the shave preparation comprises at least about 80% by weight water. In an alternate embodiment, the shave preparation comprises at least about 90% by weight water.

### Lipophilic Skin Conditioning Agent

Shave preparations of the present invention employ one or more lipophilic skin conditioning agents. The concentration level of the skin conditioning agents either singularly or collectively may range from about 1% to about 12% by weight of the base composition. Some preferred concentration levels include greater than about 1%, from about 2% to about 5%, and from about 2% to about 4%. It is to be understood that the scope of appended claims that do not specify a concentration level of the lipophilic skin conditioning agent is not limited to the levels described in this paragraph.

Exemplary skin conditioning agents include hydrocarbons, polymeric hydrocarbons, esters, ethers, and silicones selected from the group consisting of alkyl ethers, mineral oil, isoparaffin, greater than C20 hydrogenated polyisobutene, and petrolatum; and an ester composed of a branched C16-C22 alkyl chain and a mono alkyl group consisting of a linear or branched C1 to C6 alkyl chain. Some preferred skin conditioning agents comprise isostearic acid derivatives; for example, isostearyl isostearate, isopropyl isostearate, and mixtures thereof, PPG-15 Stearyl Ether and dimethicone. Other skin conditioning agents known to the skilled artisan may also be employed depending on the form of the personal care composition and the targeted skin benefit.

The skin conditioning agents may also help to reduce the coefficient of friction for personal care compositions provided herein that are in the form of shaving compositions. The reduction in friction can decrease the potential for skin irritation that can arise from contacting the skin one or more times with a razor blade. Employment of the skin conditioning agent in this context may also permit formulation flexibility regarding the type and concentration level of lubricants (as discussed more fully below) that are included in the shaving preparations.

In one embodiment, the shave preparations of the present invention is free or substantially free of any cationic skin conditioning agents, including but not limited to cationic polymers and cationic ammonium salts. Examples of such skin conditioning agents include substituted quaternary ammonium compounds (i.e., quaterniums, stearalkonium chloride, and guar hydroxypropytrimonium chloride.) See U.S. Pat. No. 4,586,650 at col. 2 line 54 et seq for additional examples of cationic skin conditioning agents which are preferably limited or not used in the present invention. Importantly, the present invention is able to achieve a desirable shave experience without requiring said cationic skin conditioning agents. This has been found to provide desired shave performance while reducing formulation costs and complexity. As defined herein, “substantially free” means that no amount of said ingredients intentionally added into the composition, but allowing for trace amounts which may be carried over from other ingredients or from processing conditions.

### Thickening Agent (Including Thickeners and Gelling Agents)

The shave preparations of the present invention contain one or more thickening agents, from about 0.1% to about 5%, alternatively from about 0.1% to about 4%, alternatively from about 0.25% to about 3%, by weight of the composition.

Nonlimiting classes of thickening agents include those selected from the following: Carboxylic Acid Polymers, Crosslinked Polyacrylate Polymers Polyacrylamide Polymers, Polysaccharides, Clays and Gums, and mixtures thereof when appropriate.

In one embodiment, compositions of the present invention include a thickening agent selected from carboxylic acid polymers, crosslinked polyacrylate polymers, polyacrylamide polymers, polysaccharides, and mixtures thereof, more preferably selected from carboxylic acid polymers, polyacrylamide polymers, polysaccharides, and mixtures thereof.

### Emulsifier

The shave preparations of the present invention contain one or more emulsifying agents, from about 0.1% to about 8%, alternatively from about 0.1% to about 5%, alternatively from about 0.25% to about 3%, by weight of the composition.

Nonlimiting examples of surfactants for emulsification for use in the compositions of the present invention are disclosed in McCutcheon's, Detergents and Emulsifiers, North American edition (1986), published by allured Publishing Corporation; and McCutcheon's, Functional Materials, North American Edition (1992). Preferred emulsifiers are nonionic surfactants/emulsifiers. Nonlimiting useful emulsifiers herein include those selected from the group consisting of alkyl glucosides, alkyl polyglucosides, polyhydroxy fatty acid amides, alkoxylated fatty acid esters, sucrose esters, alkoxylated fatty alcohols, amine oxides, and mixtures thereof.

### Lubricants

Shave compositions of the present invention may employ one or more lubricants, from about 0.1% to about 8%, alternatively from about 0.1% to about 5%, alternatively from about 0.25% to about 3%, by weight of the composition.

Exemplary lubricants include lubricous water soluble polymers, water insoluble particles, and hydrogel-forming (or water swellable) polymers, and mixtures thereof.

Useful lubricious water soluble polymers may have a molecular weight greater between about 300,000 and 15,000,000 daltons, preferably more than about one million Daltons. Nonlimiting examples of suitable lubricious water soluble polymers include polyethylene oxide, polyvinylpyrrolidone, and polyacrylamide. Nonlimiting useful water insoluble particles may include inorganic particles or organic polymer particles. Hydrogel-forming polymers are typically highly hydrophilic polymers that, in water, form organized three-dimensional domains of approximately nanometer scale. Additional polymer lubricants include: cellulose derivatives such hydroxyalkyl cellulose polymers such as hydroxyethyl cellulose and hydroxypropyl cellulose, carboxymethyl cellulose, and cellulose methyl ether and polysaccharide gums such as, for example, xanthan gum, carrageenan gum, guar gum, locust bean gum, and hydroxypropyl guar gum.

### Gel Network

In one embodiment, the shave composition is substantially free from a gel network. As used herein, the term “gel network” refers to a lamellar or vesicular solid crystalline phase which comprises at least one fatty amphiphiles. In one embodiment, the present invention contains less than about 5%, alternatively less than about 3%, alternatively less than about 1%, alternatively less than about 0.5% of at least one fatty amphiphiles. Gel networks have been found to reduce the rinse profile of these systems. Fatty alcohol gel networks have been used for years in cosmetic creams and hair conditioners. Gel networks are a re-solidified liquid crystal gel phase formed by fatty amphiphiles (e.g. cetyl or stearyl alcohol) and a hydrophilic phase (e.g. water). It is formed by undergoing a melting and then re-solidification process in the hydrophilic phase. The gel network will typically have a lower thermal transition than the melt temperature of the fatty amphiphile itself.

### Optional Ingredients

The shave preparations can further comprise additional optional ingredients. Suitable additional optional ingredients include perfume, preservatives, chelants, sensates (e.g. menthol), desquamation actives, anti-acne actives, anti-wrinkle/anti-atrophy actives, anti-oxidants/radical scavengers, flavonoids, anti-inflammatory agents, anti-cellulite agents, topical anesthetics, tanning actives, skin lightening agents, skin soothing and healing actives, antimicrobial actives, sunscreen actives, visual skin enhancers, humectants and moisturizing agents (e.g., glycerin, glycols, sorbitol) and the like. Such optional ingredients are described more fully in U.S. application Ser. No. 11/367,918, filed Mar. 3, 2006. Preferred additional optional ingredients include salicylic acid, opacifiers (e.g. mica and titanium dioxide), perfume, hydrophilic conditioning agents (e.g., glycerin) and skin sensates (e.g. menthol).

The shave preparations of the present invention may contain salicylic acid, its isomers, tautomers, salts and derivatives thereof. Alternatively, the compositions comprise from about 0.001% to about 5% salicylic acid. Alternatively, the compositions comprise from about 0.01% to about 2% salicylic acid. Alternatively, the compositions comprise from about 0.1% to about 1% salicylic acid. Without wishing to be bound by theory, it is believed that salicylic acid is efficacious for the treatment of acne on the skin. Moreover, the salicylic acid is capable of treating and/or reducing the presence of acne on the skin. Such treatment with the shave preparation of this invention involves applying the shave preparation to the skin and shaving the skin that has been treated with the shave preparation.

Dermatologically acceptable salts include alkali metal salts, such as sodium and potassium; alkaline earth metal salts, such as calcium and magnesium; non-toxic heavy metal salts; ammonium and trialkylammonium salts such as trimethylammonium and triethylammonium. Derivatives of salicylic acid include, but are not limited to, any compounds wherein the CH3 groups are individually or in combination replaced by amides, esters, amino groups, alkyls, and alcohol esters. Tautomers of salicylic acid are the isomers of salicylic acid which can change into one another with ease so that they ordinarily exist in equilibrium. Thus, tautomers of salicylic acid can be described as having the chemical formula C7H6O3 and generally having a similar structure to salicylic acid.

The compositions of the present invention may include from about 0.001% to about 5%, alternatively from about 0.01% to about 2%, and alternatively from about 0.1% to about 1%, of alpha- or beta-hydroxy acids, and derivatives, salts, isomers and tautomers thereof. Non-limiting examples of alpha- and beta-hydroxy acids include alpha-hydroxy-butyric acid, alpha-hydroxyisobutyric acid, alpha-hydroxyisocaproic acid, alpha-hydroxyisovaleric, atrolactic acid, beta-hydroxybutyric acid, beta-phenyl lactic acid, beta-phenylpyruvic acid, citric acid ethyl pyruvate, galacturonic acid, glucoheptonic acid, glucoheptono 1,4-lactone, gluconic acid, gluconolactone glucuronic acid, glucuronolactone, glycolic acid, isopropyl pyruvate, lactic acid, malic acid, amndelic acid, emthyl pyruvate, mucic acid, pyruvic acid, saccharic acid, saccharic acid 1,4-lactone, tartaric acid and tartronic acid, and mixtures thereof.

For shaving preparations that do not produce enough visual contrast between areas of the skin that have and have not been shaved, opacifiers may be added to the shaving preparation. Opacifiers may be either inorganic or organic compounds. Inorganic opacifiers include, for example, titanium dioxide, zinc oxide, talc, mica or coated mica (with oxides of titanium, tin, or iron or bismuth oxychloride), magnesium aluminum silicate, bismuth oxychloride, or other minerals. These compounds can be added as powders, dispersions, or complexes. Organic opacifiers include, for example, opaque emulsions (e.g., containing Styrene/PVP copolymer, vinyl polymers, or latexes), metal salts of amines containing 14-20 carbon atoms per molecule, alkanolamides containing 14-20 carbon atoms per molecule, organic alcohols containing 14-20 carbon atoms per molecule, insoluble salts of stearic acid, glycol mono-or distearates, propylene glycol and glycerol monostearates and palmitates. Combinations of these opacifiers can also be used. The opacifying additive is typically included in an amount of about 1 to about 6%, preferably about 2 to about 5%, by weight of the composition.

In one embodiment, the present composition comprises less than about 5% of one or more lathering surfactants, or less than about 3%, or less than about 2%, or less than about 1.5%, or less than about 1%, or less than about 0.5%. In another embodiment, the present composition is free or substantially free of lathering surfactants. A lathering surfactant is defined herein as surfactant, which when combined with water and mechanically agitated generates a foam or lather. Lathering surfactants include those selected from the group consisting of anionic lathering surfactants, amphoteric lathering surfactants, and mixtures thereof. Generally, the lathering surfactants are fairly water soluble. Examples of anionic lathering surfactants are disclosed in McCutcheon's, Detergents and Emulsifiers, North American edition (1986), published by allured Publishing Corporation; McCutcheon's, Functional Materials, North American Edition (1992); and U.S. Pat. No. 3,929,678. A wide variety of anionic lathering surfactants are useful herein. Non-limiting examples of anionic lathering surfactants include those selected from the group consisting of sarcosinates, sulfates, sulfonates, isethionates, taurates, phosphates, lactylates, glutamates, and mixtures thereof. Other anionic materials useful herein are soaps (i.e., alkali metal salts, e.g., sodium or potassium salts) of fatty acids, typically having from about 8 to about 24 carbon atoms, preferably from about 10 to about 20 carbon atoms, monoalkyl, dialkyl, and trialkylphosphate salts, alkanoyl sarcosinates. Examples of zwitterionic or amphoteric surfactants are described in U.S. Pat. Nos. 5,104,646 and 5,106,609.

### EXAMPLES

The following examples are provided to further illustrate exemplary shave preparations of the present invention. Material percentages are added neat. Activity levels are specified as needed.

TABLE 1*

| Ingredient | Ex. 1 | Ex. 2 | Ex. 3 | Ex. 4 | Ex. 5 | Ex. 6 | Ex. 7 | Ex. 8 | Ex. 9 | Ex. 10 |
|---|---|---|---|---|---|---|---|---|---|---|
| Water | Qs | Qs | Qs | Qs | Qs | Qs | Qs | Qs | Qs | Qs |
| Sepigel 305 | 2.00 | 1.60 | 2.00 | 2.00 | 1.60 | 2.00 | 2.00 | 2.00 | 2.00 | 2.00 |
| (Polyacrylamide | | | | | | | | | | |
| & C13-14 | | | | | | | | | | |
| Isoparaffin & | | | | | | | | | | |
| Laureth-7) | | | | | | | | | | |
| Polyox N12K | 0.50 | 0.50 | 0.50 | 0.50 | 0.50 | 0.50 | 0.50 | 0.50 | 0.40 | 0.50 |
| (PEG-23M) | | | | | | | | | | |
| Natrosol 250 | 0.80 | 0.50 | 0.80 | 0.80 | 0.50 | 0.80 | 0.80 | 0.90 | 0.80 | 0.80 |
| HHR (HEC) | | | | | | | | | | |
| ZPT | 0.5 | 0.5 | 0.5 | 2 | 2 | 2 | 2 | 1 | 1 | 1 |
| Glycerin | — | — | — | — | — | — | 5.00 | — | — | 5.00 |
| Brij 35 | 2.00 | 2.00 | 2.00 | 2.00 | 2.00 | 2.00 | 2.00 | 2.00 | 2.00 | 2.00 |
| (Laureth-23, | | | | | | | | | | |
| 100% Active) | | | | | | | | | | |
| Salicylic Acid | — | — | — | — | — | 0.50 | 0.50 | 0.50 | 0.50 | — |
| Arlamol E | 2.00 | 2.00 | 2.00 | — | — | 2.00 | 2.00 | 2.00 | 2.00 | 2.00 |
| (PPG-15 | | | | | | | | | | |
| Stearyl Ether) | | | | | | | | | | |
| Niacinamide | — | — | — | — | — | — | — | — | — | 2.50 |
| Acusol OP301 | — | — | — | — | — | — | 2.50 | 2.50 | 2.50 | — |
| Opacifier | | | | | | | | | | |
| (40% solids) | | | | | | | | | | |
| (Water and | | | | | | | | | | |
| Styrene/Acrylic | | | | | | | | | | |
| Copolymer) | | | | | | | | | | |
| DC 1503 | — | — | — | 2.00 | 2.00 | — | — | — | — | — |
| Expancel | — | — | — | — | — | — | — | — | 2.00 | — |
| 920-WE40 | | | | | | | | | | |
| D24 | | | | | | | | | | |
| Disodium | 0.10 | 0.10 | 0.30 | 0.10 | 0.10 | 0.10 | 0.05 | 0.10 | — | 0.10 |
| EDTA (EDETA | | | | | | | | | | |
| BD) | | | | | | | | | | |
| Perfume 1 | 0.15 | 0.15 | 0.15 | 0.15 | 0.15 | — | — | — | — | 0.15 |
| Perfume 2 | — | — | — | — | — | — | — | — | — | — |
| Perfume 3 | — | — | — | — | — | 0.15 | 0.15 | — | — | — |
| Perfume 4 | — | — | — | — | — | — | — | 0.50 | 0.50 | — |
| Glydant Plus | 0.20 | 0.20 | 0.20 | 0.20 | 0.20 | 0.20 | 0.20 | 0.40 | 0.40 | 0.20 |

| Ingredient | Ex. 11 | Ex. 12 | Ex. 13 | Ex. 14 | Ex. 15 | Ex. 16 |
|---|---|---|---|---|---|---|
| Water | Qs | Qs | Qs | Qs | Qs | Qs |
| Cetyl Alcohol | — | — | — | 3 | 3 | 3 |
| Sepigel 305 | 2 | 2 | 2 | 1 | 1 | 1 |
| (Polyacrylamide | | | | | | |
| & C13-14 | | | | | | |
| Isoparaffin & | | | | | | |
| Laureth-7) | | | | | | |
| Polyox N12K | 0.6 | 0.8 | 0.8 | 0.8 | 0.8 | 0.8 |
| (PEG-23M) | | | | | | |
| Natrosol 250 | 0.8 | 0.8 | 0.8 | 0.8 | 0.8 | 0.8 |
| HHR (HEC) | | | | | | |
| Glycerin | — | — | — | — | 2 | — |
| Plantaren 1200 | 4 | 4 | 8 | 4 | 4 | 8 |
| N UP (Lauryl | | | | | | |
| Glucoside, | | | | | | |
| 50% Active) | | | | | | |
| Sodium Lauryl | — | — | — | 0.5 | 0.5 | 0.5 |
| Sulfate (29% Active) | | | | | | |
| Arlamol E (PPG-15 | 2 | 2 | 2 | 2 | 2 | 2 |
| Stearyl Ether) | | | | | | |
| Acusol OP301 | 2.5 | 2.5 | — | — | — | — |
| Opacifier (40% solids) | | | | | | |
| (Water and Styrene/ | | | | | | |
| Acrylic Copolymer) | | | | | | |
| Expancel 920- | 2 | 2 | 2 | 2 | 2 | 2 |
| WE40 D24 | | | | | | |
| Citric Acid | 0.06 | 0.06 | 0.15 | 0.06 | 0.12 | 0.2 |
| Menthol | _{—} | 0.05 | 0.05 | 0.05 | 0.05 | 0.065 |
| Glydant Plus | 0.3 | 0.3 | 0.3 | 0.3 | 0.3 | 0.3 |
| ZPT | 0.2 | 0.5 | 0.7 | 1.0 | 1.2 | 1.5 |

| Ingredient | Ex. 17 | Ex. 18 | Ex. 19 |
|---|---|---|---|
| Water | Qs | Qs | Qs |
| Cetyl Alcohol | 3 | 3 | 3 |
| Sepigel 305 | 1 | 2 | 2 |
| (Polyacrylamide & | | | |
| C13-14 Isoparaffin | | | |
| & Laureth-7) | | | |
| Polyox N12K (PEG-23M) | 0.8 | 0.8 | 0.8 |
| Natrosol 250 HHR (HEC) | 0.8 | 0.8 | 0.8 |
| Glycerin | 2 | — | — |
| Brij 35 (Laureth-23, | — | — | — |
| 100% Active) | | | |
| Plantaren 1200 N UP | 8 | 1 | 1 |
| (Lauryl Glucoside, | | | |
| 50% Active) | | | |
| Lenocare SMS-30 | — | 7 | 7 |
| (Sodium Myristoyl | | | |
| Sarcosinate, 30% | | | |
| Active) | | | |
| Mackam DAB-ULS | — | — | 6 |
| (Lauramidopropyl | | | |
| Betaine, 35% Active) | | | |
| Sodium Lauryl Sulfate | 0.5 | 0.5 | 0.5 |
| (29% Active) | | | |
| Arlamol E (PPG-15 | | 2 | 2 |
| Stearyl Ether) | | | |
| Petrolatum | 2 | — | — |
| Expancel 920-WE40 | 2 | 2 | 2 |
| D24 | | | |
| Citric Acid | 0.2 | 0.18 | 0.18 |
| Menthol | — | 0.05 | 0.05 |
| Perfume 4 | 0.5 | 0.6 | 0.6 |
| Glydant Plus | 0.3 | 0.3 | 0.3 |
| ZPT | 3 | 4 | 5 |

*Percentages in Table 1 (Examples 1-19) are on a weight by weight basis.

Qs, as used herein means the remaining amount to reach 100%. The above Examples are made according to the method below.
1. Weigh out the water in a vessel sufficient to hold the entire batch
2. Insert an overhead mixer with impeller into the vessel and increase agitation to create a vortex
3. Pre-blend the thickener and polymer powders
4. Sprinkle the polymer blend into the vortex until incorporated
5. Begin heating batch to 70C to hydrate the polymers
6. Once the batch is at 70C, add the oil and mix until uniform and dispersed
7. Add the liquid dispersion polymer to the batch and mix until uniform and hydrated, increasing rpms to maintain good mixing
8. Add the surfactant and mix until uniform and dispersed
9. Begin cooling batch to below 45C
10. Once below 45C, add the perfume, preservatives and other temperature-sensitive additives
11. Cool to below 35C and QS with water

For product with acne control actives, weigh out the solubilizing agent and salicylic acid in a separate vessel and mix until dissolved. Add to the batch during STEP 6 addition.

For product with water-soluble actives, add to the batch during STEP 10 additions

For product with oil-soluble actives, add to the batch during STEP 6 additions

For product with improved tracking, add the opacifier during the STEP 10 additions

For product with lower density, add the Expancel to the batch during STEP 6 additions

While the specification concludes with the claims particularly pointing and distinctly claiming the invention, it is believed that the present invention will be better understood from the following description.

The devices, apparatuses, methods, components, and/or compositions of the present invention can include, consist essentially of, or consist of, the components of the present invention as well as other ingredients described herein. As used herein, “consisting essentially of” means that the devices, apparatuses, methods, components, and/or compositions may include additional ingredients, but only if the additional ingredients do not materially alter the basic and novel characteristics of the claimed devices, apparatuses, methods, components, and/or compositions.

All percentages and ratios used herein are by weight of the total composition and all measurements made are at 25° C., unless otherwise designated. A degree is a planar unit of angular measure equal in magnitude to 1/360 of a complete revolution.

All measurements used herein are in metric units unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A shave preparation comprising:
water;
one or more lipophilic skin conditioning agents;
one or more thickening agents;
one or more emulsifying agents;
one or more lubricants, and
from about 0.01% to 5% of an anti-irritation agent comprising a pyrithione. a polyvalent metal salt of pyrithione, and a mixture thereof, and optionally an additional malodor control agent.

2. The shaving preparation of claim 1, wherein said anti-irritation agent comprises zinc pyrithione.

3. The shaving preparation claim 2, wherein said zinc pyrithione is in the form of a platelet having a median particle diameter of about 0.5 microns to about 10, a mean particle diameter of about 0.5 to about 10 microns, and a thickness of about 0.6 to about 15 microns.

4. The shaving preparation of claim 3, wherein the zinc pyrithione is in platelet particle form having an average size of up to about 20 μm.

5. The shaving preparation of claim 3, further comprising from 0.001% to 10% of a zinc salt.

6. The shaving preparation of claim 6, wherein said zinc salt comprises zinc oxide.

7. The shave preparation of claim 1, wherein the shave preparation comprises from about 1% to about 12% by weight of the one or more lipophilic skin conditioning agents.

8. The shave preparation of claim 1, wherein the shave preparation comprises from about 0.1% to about 5% by weight of the one or more thickening agents.

9. The shave preparation of claim 1, wherein the shave preparation comprises from about 0.1% to about 8% by weight of the one or more emulsifying agents.

10. The shave preparation of claim 1, wherein the shave preparation comprises from about 0.1% to about 8% by weight of the one or more lubricants.

11. The shave preparation of claim 1, wherein the shave preparation comprises from about 0.001% to about 5% by weight of the salicylic acid.

12. The shave preparation of claim 1, wherein the shave preparation comprises from about 1 to about 6%, by weight of the opacifier.

13. The shave preparation of claim 1, substantially free of a cationic skin conditioning agent.

14. The shave preparation of claim 1, comprising less than 2% of a lathering surfactant.

15. A shave preparation comprising:
water;
one or more lipophilic skin conditioning agents;
one or more thickening agents;
one or more emulsifying agents;
one or more lubricants; and
an anti-irritation agent comprising a zinc pyrithione,
wherein the shave preparation is substantially free of a gel network.

16. The shave preparation of claim 15, wherein the shave preparation comprises from about 1% to about 12% by weight of the one or more lipophilic skin conditioning agents.

17. The shave preparation of claim 15, wherein the shave preparation comprises from about 0.1% to about 5% by weight of the one or more thickening agents.

18. The shave preparation of claim 15, wherein the shave preparation comprises from about

0. 1% to about 8% by weight of the one or more emulsifying agents.

19. The shave preparation of claim 15, wherein the shave preparation comprises from about 0.1% to about 8% by weight of the one or more lubricants.

20. A shave preparation comprising:
at least 60% water;
from about 2% to about 4% of one or more lipophilic skin conditioning agents;
from about 0.25% to about 3% of one or more thickening agents;
from about 0.25% to about 3% of one or more emulsifying agents;
from about 0.25% to about 3% of one or more lubricants;
from about 0.25% to about 2.5% of a zinc pyrithione in the form of a platelet having a median particle diameter of about 0.5 microns to about 10, a mean particle diameter of about 0.5 to about 10 microns, and a thickness of about 0.6 to about 15 microns, and
wherein the shave preparation is substantially free of a gel network.
